(19) United States
(12) Patent Application Publication
(10) Pub. No.: US 2008/0097168 A1
(43) Pub. Date: Apr. 24, 2008

(54) MEDICAL WORKSTATION WITH A THERAPY MODULE

(75) Inventors: Hermann HOPERMANN, Badendorf (DE); Jurgen MANIGEL, Scharbeutz-Klingberg (DE); Kay FRENSCH, Scharbeutz-Klingberg (DE); Jens KOHNE, Lubeck (DE); Claus BUNKE, Sereetz (DE); Gerald PANITZ, Klenzau (DE); Martin WUNDERLICH, Lubeck (DE); Dieter SETTGAST, Lubeck (DE); Volker SCHIERSCHKE, Lubeck (DE)

Correspondence Address: MCGLEW & TUTTLE, PC, P.O. BOX 9227, SCARBOROUGH STATION, SCARBOROUGH, NY, 10510-9227, US

(73) Assignee: DRAGER MEDICAL AG & CO. KG, Lubeck (DE)

(21) Appl. No.: 11/873,642

(22) Filed: Oct. 17, 2007

(30) Foreign Application Priority Data
Oct. 24, 2006 (DE) 10 2006 049 982.4

(51) Int. Cl. A61B 5/00

(52) U.S. Cl. 600/300

(57) ABSTRACT

A medical workstation has a control and display unit (1) connected to a patient monitor module (2) via a communications line (5). The medical workstation has an interface accessible to the user for connecting a therapy module (3). The control and display unit (1) has a microprocessor and the therapy module (3) has at least one microprocessor and a nonvolatile memory. The control and display unit (1) has an area (6) that is visible to the user and can be operated by the user with graphic elements for the display and modification of therapy settings. The control and display unit (1) has an operating program, which is designed such that the area (6) on the control and display unit (1) can be operated by the user only in case a compatible therapy module (3) is connected.

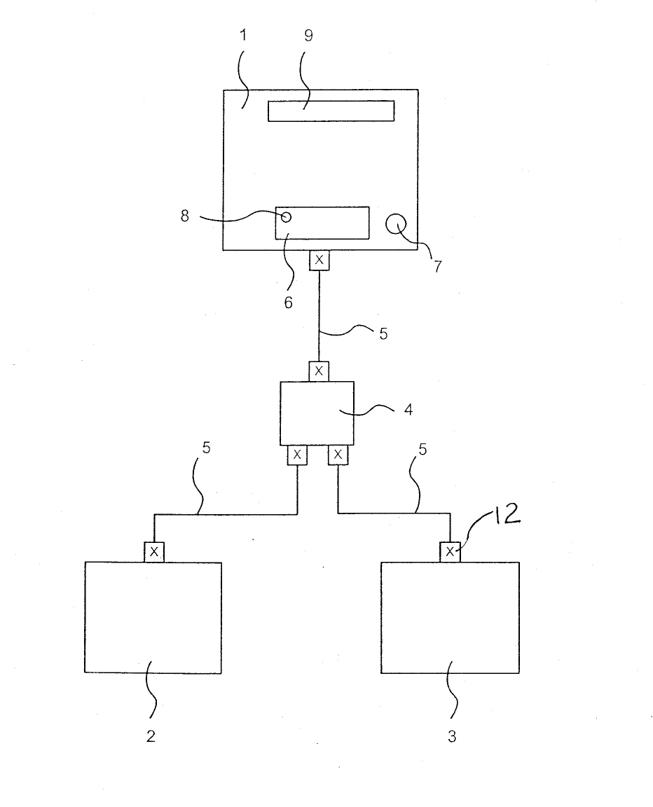

### CROSS REFERENCE TO RELATED APPLICATIONS

This application claims the benefit of priority under 35 U.S.C. §119 of German Patent Application DE 10 2006 049 982.4 filed Oct. 24, 2006, the entire contents of which are incorporated herein by reference.

### FIELD OF THE INVENTION

The present invention pertains to a medical workstation with a therapy module.

### BACKGROUND OF THE INVENTION

Patient monitors or patient monitor modules, which detect, display and monitor diverse physiological parameters of the patient, are used in intensive care units in hospitals. These parameters include, for example, the electrocardiogram (ECG), invasively and non-invasively measured blood pressures, body temperatures, the oxygen saturation of the blood and the carbon dioxide concentration in the breathing gas.

The patient monitors used have a control and display unit, among other things, for the graphic or numeric display of the measured parameters and for displaying alarms.

Moreover, the user has the possibility of configuring the patient monitor via this control and display unit. Examples of essential configurations are alarm limits, display screen layout, and parameter settings.

Besides patient monitors, which process only physiological parameters of the patient and can therefore be called passive devices, other active medical devices, devices also called therapy modules, which in turn assume therapeutic functions, are used as well, depending on the clinical picture. These include, among other things, syringe and infusion pumps, respirators and thermotherapy devices.

Respirators support the patient's spontaneous breathing or replace it completely. These also have, as a rule, a control and display unit of their own.

Syringe and infusion pumps supply the patient with drugs, nutrition or other solutions via an intravenous access and likewise have a control and display unit, which is usually integrated within the housing of the pump.

Thermotherapy devices support the heat regulation of premature infants and offer a defined climate in terms of air temperature, humidity and oxygen content of the air in the form of closed incubators. Thermotherapy devices also have a control and display unit of their own, usually integrated within the housing of the device.

The presence of a plurality of control and display units is associated with drawbacks. For example, these cause costs, must be positioned at the bedside of the patient being treated and have, as a rule, different control concepts. The latter leads to a corresponding effort in terms of training the users and increases the probability of operating errors.

Devices that integrate the function of a patient monitor and the function of therapy devices on one control and display unit circumvent these drawbacks. Thus, devices that integrate the function of a patient monitor and of a respirator on one control and display unit are now commercially available. These are basically respirators, linked with the option of using their control and display unit also as a control and display unit of a patient monitor. The fact that the control and display unit cannot be operated without the presence of the components for the respiration function has proved to be a drawback.

The fact that practically all patients of an intensive care unit are connected to a patient monitor, but frequently only about half of the patients are also respirated on a respirator gives rise to the need for the ability to scale the device configuration in a more flexible manner in conjunction with a central control and display unit.

This aspect is taken up again in the patent applications WO 2005/050524 A2 (Modular Medical Care System) and WO 2005/050523 A2 (Processing Device and Display System). The presence of modules for monitoring, ventilation, infusion, anesthesia, which can be configured into a medical workstation in a flexible manner and have a shared control and display unit, is an essential element of the system described there. It is mentioned in the patent applications that it is possible to add or remove individual modules into and from the system also during the operation of other modules.

### SUMMARY OF THE INVENTION

The object of the present invention is to provide a device to make it possible to integrate a therapy module with or without a control and display unit of its own in a medical workstation currently in operation with a central control and display unit.

According to the invention, a medical workstation is provided with a control and display unit connected to a patient monitor module via a communications line. The medical workstation has an interface accessible to the user for connecting a therapy module. The control and display unit has a microprocessor and the therapy module has at least one microprocessor and a nonvolatile memory. The control and display unit has an area with graphic elements for the display and modification of therapy settings, which area is visible to the user and is able to be operated by same. The control and display unit has an operating program, which is designed such that the area on the control and display unit is able to be operated by the user only in case of connection of a compatible therapy module.

The interface for connecting the therapy module may be located at a central network element.

The control and display unit may include at least one display area, in which alarms, warnings or instructions pertaining to the therapy module are displayed.

The therapy module may have a microprocessor, which starts operating after being connected to the control and display unit.

The interface for the communications line of the therapy module may have a mechanical securing means, so that accidental disconnection of the therapy module is ruled out.

The area may contain graphic elements for displaying and modifying therapy settings.

The area may also contain graphic elements for performing a test of the therapy module.

The area may also contain graphic elements for starting and stopping the therapy.

The following problems are solved with the invention:

Preparation/test of the therapy module;

Behavior during connection/disconnection of the therapy module;

Compatibility of software versions;

Start/shutdown behavior;

Dynamic adaptation of the graphic control surface.

An exemplary embodiment of the present invention will be explained below by means of the only figure. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

The only figure schematically shows the combination of the components of the medical workstation according to the invention.

### DESCRIPTION OF THE PREFERRED EMBODIMENT

Referring to the drawings in particular, a medical workstation according to the invention has a central control and display unit 1, which has at least one display area 9 for displaying alarms, warnings and instructions. The control and display unit 1 is connected to a patient monitor module 2 via a communications line 5. The control and display unit 1 is preferably connected to the patient monitor module 2 via a central network element 4 (switch/hub), which makes possible a star-shaped network topology. The medical workstation has an interface for connecting a therapy module 3, which interface is readily accessible to the user. The interface for connecting the therapy module 3 is preferably located at the central network element 4. The therapy module 3 has at least one microprocessor as well as a nonvolatile memory. The results of the most recent testing of the module, i.e., for example, actuator tests, sensor tests, tightness test, alarm tone test, calibration and compensation values, compliance and resistance of the tubing system are stored in the nonvolatile memory of the therapy module 3. The control and display unit 1 has a microprocessor.

To integrate the therapy module 3 within the medical workstation, the module is connected by the user to the system via a communications line 5. If the therapy module 3 has no control and display unit of its own, at least one microprocessor (main microprocessor) is turned off before the therapy module 3 is connected to the system. A second microprocessor (auxiliary microprocessor) is preferably running, and this is supplied from a battery of the therapy module 3. The communications line 5 of the therapy module 3 or the interface intended for this has especially a mechanical securing means 12 in order to prevent accidental disconnection of the therapy module 3. This securing means 12 snaps in when the therapy module 3 is being connected to the system. Disconnection of the therapy module 3 is now possible only by actuating the mechanical securing means 12. After connecting the therapy module 3, the module is recognized by the control and display unit 1. If the therapy module 3 has no control and display unit of its own, this preferably takes place by communication between the auxiliary microprocessor of the therapy module 3 and the control and display unit 1. Immediately after the connection to the therapy module 3 has been recognized, the control and display unit 1 shows an instruction in the display area 9, which informs the user that a therapy module 3 was recognized in the system. If the therapy module 3 has no control and display unit of its own, at least one microprocessor, preferably the main microprocessor, starts its operation in this therapy module 3 after the connection of the therapy module 3. After the therapy module 3 has been connected, this therapy module 3 sends its program version to the control and display unit 1. As an alternative, it is possible that the control and display unit 1 sends its program version to the therapy module 3.

After the program version has been sent, the control and display unit 1 checks the compatibility of its program version with that of the therapy module 3. It is optionally also possible that the therapy module 3 checks the compatibility of its program version with that of the control and display unit 1 and sends the result to the control and display unit 1. In case of incompatibility of the two program versions, the control and display unit 1 shows in the display area 9 an instruction that informs the user that a program incompatibility was recognized.

In case of compatibility of the two program versions, an area 6 becomes visible and able to be operated by the user on the control and display unit 1. As an alternative, this area 6 is always visible, but it becomes able to be operated for the user only in case of compatibility of the two program versions. Area 6 contains graphic elements for performing a function test of the therapy module 3. This test comprises, for example, actuator tests, sensor tests, tightness test, alarm tone test, the determination of calibration and compensation values, the determination of the compliance and resistance of the tubing system. The control knob 7 is used to modify and acknowledge settings. The area 6 also contains graphic elements 8 for displaying and modifying therapy settings. Examples of therapy settings are:

1. For respiration therapy: Respiration mode, tidal volume, respiration rate, inspiration pressure, positive end expiratory pressure (PEEP), inspiration time, percentage of oxygen in the breathing air, and trigger settings.

2. For infusion therapy: Type of syringe, drug, infusion volume, delivery rate, delivery rate rise time, delivery rate decline time, and bolus rate.

3. For thermotherapy: Air temperature, humidity, oxygen content in the air.

In case of a respirator as a therapy module 3, area 6 contains especially graphic elements for input of the configuration of the tubing system being used and the breathing gas humidifier. Area 6 also contains graphic elements for starting and stopping the therapy. In case of compatibility of the two program versions of the control and display unit 1, on the one hand, and the therapy module 3, on the other hand, the control and display unit 1 activates the availability of operating elements for additional functions, for example, for maneuvers such as “Inspiration Hold”, “Expiration Hold”, “P0.1” pressure measurement, which are bound to the presence of a therapy module 3. However, as an alternative, these operating elements may also be located outside the visible area 6 of the control and display unit 1.

After the therapy has been started by the user, the control and display unit 1 automatically displays selected real time curves of measured values of therapy parameters.

After the therapy has been stopped by the user, the control and display unit 1 automatically replaces the display of selected real-time curves and measured values of therapy parameters with the display of other parameters. If the communications line 5 of the therapy module 3 is pulled by the user after stopping the therapy, the control and display unit 1 shows an instruction in the display area 9, which informs the user that the therapy module 3 was disconnected. If the communications line 5 of the therapy module 3 is disconnected after the therapy had been stopped by the user, at least one microprocessor of the therapy module 3, preferably the main microprocessor, is turned off if the therapy module has no control and display unit of its own, and area 6 on the control and display unit 1 becomes invisible for the user. Area 6 remains visible in an alternative variant, and it is deactivated in case of disconnection, i.e., it becomes unable to be operated any longer. If the communications line 5 of the therapy module 3 is disconnected after the therapy had been stopped by the user, the control and display unit 1 additionally deactivates the availability of operating elements for other functions, for example, the above-described maneuvers such as “Inspiration Hold”, “Expiration Hold”, “P0.1” pressure measurement, which are bound to the presence of a therapy module 3. However, these operating elements may, in principle, also be located outside the visible area 6.

It is possible with the system being described to set up the therapy module 3 outside the immediate area of the patient by storing the results of the module test in a nonvolatile memory in the therapy module 3, so that therapy is available faster at the bedside of the patient. It is also possible to perform the self-test even after the connection to the system.

The start-up/shutdown of a therapy module 3 without a control and display unit of its own takes place only on connection/disconnection of the communications line 5 to/from the system, with the consequence of reduced energy demand for the therapy module 3 when this is not connected to the system. The dynamic display of the operating elements for a therapy module 3 on a central control and display unit 1 advantageously takes place as a function of the availability of the therapy module, so that optimal utilization of the available area of the control and display unit 1 is guaranteed. Another advantage is the early recognition of incompatible program versions and the fact that software compatibility is guaranteed. Finally, an accidental disconnection of the therapy module 3 is avoided.

While specific embodiments of the invention have been shown and described in detail to illustrate the application of the principles of the invention, it will be understood that the invention may be embodied otherwise without departing from such principles.

What is claimed is:

1. A medical workstation comprising:
a control and display unit with a microprocessor;
a patient monitor module;
a communications line, said control and display unit being connected to said patient monitor module via said communications line;
a therapy module with a microprocessor and a nonvolatile memory;
an interface accessible to the user for connecting said therapy module;
an area with graphic elements for the display and modification of therapy settings of said therapy module, said area with graphic elements being provided by said control and display unit with said area with graphic elements being visible to a medical workstation user and disposed for operation by the medical workstation user;
an operating program of said control and display unit for allowing said area with graphic elements on said control and display unit to be operated by the medical workstation user only in case of connection of a compatible therapy module to said control and display unit.

2. A medical workstation in accordance with claim 1, further comprising:
a central network element of a network connected to said communications line, said interface for connecting said therapy module being located at said central network element.

3. A medical workstation in accordance with claim 1, wherein said control and display unit has at least one display area, in which alarms, warnings or instructions pertaining to said therapy module are displayed and/or issued.

4. A medical workstation in accordance with claim 1, wherein said microprocessor of said therapy module starts operating after being connected to said control and display unit.

5. A medical workstation in accordance with claim 2, wherein said interface for said communications line of said therapy module has a mechanical securing means, so that accidental disconnection of said therapy module is prevented.

6. A medical workstation in accordance with claim 1, wherein said area with graphic elements includes graphic elements for displaying and modifying therapy settings.

7. A medical workstation in accordance with claim 1, wherein said area with graphic elements includes graphic elements for performing a test of said therapy module.

8. A medical workstation in accordance with claim 1, wherein said area with graphic elements includes graphic elements for starting and stopping the therapy.

9. A medical workstation comprising:
a patient monitor module;
a therapy module with a microprocessor and a nonvolatile memory;
a control and display unit with a microprocessor and an area with graphic elements for the display and modification of therapy settings of said therapy module, said area with graphic elements being provided by said control and display unit with said area with graphic elements being visible to a medical workstation user and disposed for operation by the medical workstation user;
a network connection interface with communications lines connecting said control and display unit to said patient monitor module and to said therapy module;
an operating program associated with said control and display unit for allowing said area with graphic elements on said control and display unit to be operated by the medical workstation user only in case of connection of a compatible therapy module to said control and display unit.

10. A medical workstation in accordance with claim 9, wherein said network connection interface includes: a central network element of a network connected to said communications lines; and an interface for connecting said therapy module, said interface being located at said central network element.

11. A medical workstation in accordance with claim 10, wherein said control and display unit has at least one display area, in which alarms, warnings or instructions pertaining to said therapy module are displayed and/or issued.

12. A medical workstation in accordance with claim 10, wherein said microprocessor of said therapy module starts operating after being connected to said control and display unit.

13. A medical workstation in accordance with claim 10, wherein said interface for said therapy module has a mechanical securing means, said securing means preventing accidental disconnection of said therapy module.

14. A medical workstation in accordance with claim 10, wherein said area with graphic elements includes graphic elements for displaying and modifying therapy settings.

15. A medical workstation in accordance with claim 10, wherein said area with graphic elements includes graphic elements for performing a test of said therapy module.

16. A medical workstation in accordance with claim 10, wherein said area with graphic elements includes graphic elements for starting and stopping the therapy.

17. A method of establishing a medical workstation, the method comprising the steps of:
providing a patient monitor module;
providing a therapy module with a microprocessor and a nonvolatile memory;
providing a control and display unit with a microprocessor and an area with graphic elements for the display and modification of therapy settings of said therapy module, said area with graphic elements being provided by said control and display unit with said area with graphic elements being visible to a medical workstation user and disposed for operation by the medical workstation user;
providing a network connection interface with communications lines connecting said control and display unit to said patient monitor module and to said therapy module;
providing an operating program associated with said control and display unit;
using said program for allowing said area with graphic elements on said control and display unit to be operated by the medical workstation user only in case of connection of a compatible therapy module to said control and display unit.

18. A method of establishing a medical workstation in accordance with claim 17,wherein said network connection interface includes: a central network element of a network connected to said communications lines; and an interface for connecting said therapy module, said interface being located at said central network element wherein said interface for said therapy module has a mechanical securing means, so that accidental disconnection of said therapy module is prevented.

19. A method of establishing a medical workstation in accordance with claim 18, wherein said control and display unit has at least one display area, in which alarms, warnings or instructions pertaining to said therapy module are displayed and/or issued.

20. A method of establishing a medical workstation in accordance with claim 17, wherein:
said area with graphic elements includes graphic elements for displaying and modifying therapy settings;
said area with graphic elements includes graphic elements for performing a test of said therapy module; and
said area with graphic elements includes graphic elements for starting and stopping the therapy.
